(19) United States
(12) Patent Application Publication
(10) Pub. No.: US 2025/0144379 A1
(43) Pub. Date: May 8, 2025

(54) BALLOON CATHETER WITH CONTACT INDICATOR AND RELATED METHODS

(71) Applicant: BARD PERIPHERAL VASCULAR, INC., Franklin Lakes, NJ (US)

(72) Inventors: Samuel Shreve, Scottsdale, AZ (US); Cyal Lecy, Buffalo, MN (US)

(21) Appl. No.: 18/837,867

(22) PCT Filed: Feb. 11, 2022
(86) PCT No.: PCT/US2022/016112

§ 371 Date: Aug. 12, 2024

(51) Int. Cl. A61M 25/10

(52) U.S. Cl. CPC A61M 25/10187

(57) ABSTRACT

A medical apparatus for performing a medical procedure in a vessel having an inner wall. The apparatus includes an inflatable balloon incorporating a pressure sensor for sensing contact between the inner wall of the vessel and the balloon when inflated. Related methods are also disclosed.

### BACKGROUND OF THE INVENTION

Current balloon catheters used for dilatation of a vessel require the user to rely on imaging or fluoroscopy to ensure the balloon upon inflation is contacting the vessel wall sufficiently to provide the desired treatment effect. This can require a significant amount of guesswork. This can be especially troublesome in the case of drug-coated balloons, since contact with the vessel wall is necessary to ensure effective delivery of the drug.

Accordingly, a need is identified for a manner of providing reliable feedback to the user as to whether the balloon is contacting the vessel wall.

### SUMMARY OF THE INVENTION

An object of the disclosure is to provide a balloon with a pressure sensor for indicating contact with an inner wall of a vessel during dilatation, as well as an indicator that provides reliable feedback to the user as to whether the balloon (or potentially any portion thereof) is contacting the vessel inner wall based on the pressure sensor. The resulting apparatus may be especially useful in connection with drug-coated balloons by confirming that reliable contact is established to ensure transfer of the drug to the vessel inner wall as desired or intended.

According to a first aspect of the disclosure, a medical apparatus for performing a medical procedure in a vessel having an inner wall comprises an inflatable balloon including a pressure sensor for sensing contact between the inner wall of the vessel and the balloon when inflated.

In one embodiment, the pressure sensor comprises a first conductor for contacting a second conductor to form an electrical connection when the balloon is compressed against the inner wall. The first conductor may be connected to a source of power, such as a battery. The second conductor may comprise a band, and the second conductor may comprise an elongated strip for contacting the first conductor.

In one embodiment, the first conductor is located at a proximal end portion of the inflatable balloon. A third conductor may be located at a distal end portion of the inflatable balloon for contacting the second conductor when the balloon is inflated to contact an inner wall of the vessel. The third conductor may be located along an intermediate portion of the balloon for contacting the second conductor when the balloon is inflated to contact an inner wall of the vessel.

The first and second conductors may be provided in a wall of the balloon. The wall may include an insulator for separating the first and second conductors when the balloon is not in contact with the inner wall of the vessel.

An indicator may be provided for indicating when the first and second conductors are in contact. A shaft for supporting the balloon may include a proximal hub including the indicator or a transmitter (wireless or wired) for transmitting a signal indicating an electrical connection between the first and second conductors. An inflator for inflating the balloon may instead include the indicator or transmitter.

The balloon may include a plurality of first conductors, the indicator indicating when each of the first conductors forms an electrical connection with the second conductor.

In alternative forms, the pressure sensor comprises a pocket of fluid within a wall of the balloon. A further alternative is a piezoelectric material connected to a wall of the balloon.

In accordance with another aspect of the disclosure, a medical apparatus for performing a medical procedure in a vessel having an inner wall. The apparatus comprises an inflatable balloon comprising a first sensor for a first portion of the balloon for sensing contact between the first portion of the balloon when inflated and the inner wall of the vessel. A second sensor is provided for a second portion of the balloon for sensing contact between the second portion of the balloon when inflated and the inner wall of the vessel.

In one embodiment, the first sensor is located along a proximal end portion of the balloon and the second sensor is located along a distal end portion of the balloon. A third sensor between the first and second sensors for sensing contact between a third portion of the balloon when inflated and the inner wall of the vessel. The first sensor comprises a first conductor for contacting a second conductor to form an electrical connection when the first portion balloon is inflated to contact the inner wall of the vessel. The second sensor comprises a third conductor for contacting the second conductor to form an electrical connection when the second portion balloon is inflated to contact the inner wall of the vessel.

According to a third aspect of the disclosure, a medical apparatus for performing a medical procedure in a vessel having an inner wall is provided. The apparatus comprises an inflatable balloon comprising a first sensor along a first portion of the balloon for sensing contact between the first portion of the balloon when inflated and the inner wall of the vessel. An indicator may also be provided for indicating when the first and second portions of the inflatable balloon are in contact with the inner wall of the vessel.

In one embodiment, an associated catheter shaft includes a proximal hub including the indicator, or adapted for transmitting a signal from the first sensor (such as wirelessly). The first sensor may be located along a proximal end portion of the balloon and further including a second sensor is located along a distal end portion of the balloon. A third sensor may be provided between the first and second sensors for sensing contact between a third portion of the balloon when inflated and the inner wall of the vessel.

The first sensor may comprise a first conductor for contacting a second conductor to form an electrical connection when the first portion balloon is inflated to contact the inner wall of the vessel. A second sensor comprising a third conductor may be provided for contacting the second conductor to form an electrical connection when the second portion balloon is inflated to contact the inner wall of the vessel.

Still a further aspect of the disclosure pertains to a method for evaluating an inner wall of a vessel. The method comprises expanding a medical balloon including at least one sensor for sensing contact between a portion of the balloon and the inner wall, and mapping the inner wall based on the contact sensed by the at least one sensor.

In one embodiment, the mapping comprises visualizing the inner wall of the vessel. The visualizing may comprise visualizing the inner wall of the vessel in a first inflation state of the medical balloon and visualizing the inner wall of the vessel in a second inflation state of the balloon.

Yet another aspect of the disclosure pertains to a method of performing a medical procedure in a vessel having an inner wall. The method comprises inflating a balloon in the vessel, and indicating contact between a plurality of portions of the balloon and the inner wall of the vessel.

In one embodiment, the indicating step comprises providing a first visual indication of contact between a first portion of the balloon and the inner wall, and a second visual indication of contact between a second portion of the balloon and the inner wall. The step of providing the first and second visual indications comprises illuminating first and second lights on an indicator attached to a shaft supporting the balloon.

### BRIEF DESCRIPTION OF THE DRAWINGS

The above and further advantages of the present disclosure may be better understood by referring to the following description in conjunction with the accompanying drawings in which:

FIG. 1 is a perspective view of a balloon catheter.

FIG. 1A is a cross-sectional view along line 1A-1A of FIG. 1.

FIG. 2 is a partially cutaway perspective view of a balloon catheter including a pressure sensor.

FIG. 2A is a cross-sectional view of a wall of the balloon of FIG. 2.

FIG. 3 is a cutaway top view of a proximal hub for a balloon catheter including an indicator for indicating contact with an inner wall of the vessel.

FIG. 4 is a partially cutaway view of an indeflator including an indicator for indicating contact with an inner wall of the vessel.

FIG. 5 is a cutaway top view of a proximal hub for a balloon catheter with a pressure sensor and a transmitter for displaying indicating contact with an inner wall of the vessel.

FIG. 6 is another cutaway top view of a proximal hub for a balloon catheter with a pressure sensor and a transmitter for displaying indicating contact with an inner wall of the vessel.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the disclosed inventive concepts.

The invention of this disclosure is not limited in its application to the details of construction and the arrangement of the components set forth in the following description or illustrated in the drawings. The inventive concepts disclosed are capable of other embodiments or of being practiced or carried out in various ways. It is also to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable subcombination.

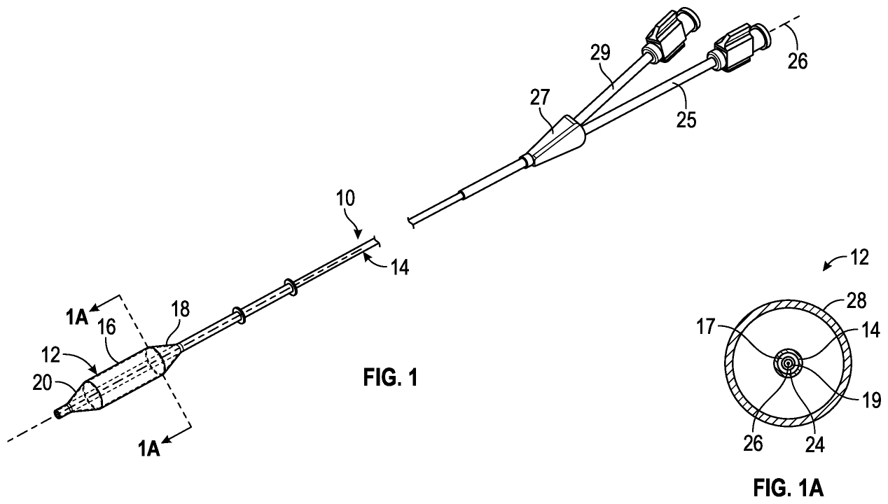

With reference to FIG. 1, provided is a catheter 10 having a distal end portion including an expandable element in the form of a single balloon 12 mounted on a catheter shaft 14. The balloon 12 includes an intermediate portion 16, or “barrel,” and end portions 18, 20. In one embodiment, the end portions 18, 20 of the balloon 12 reduce in diameter or taper to join the intermediate portion 16 to the catheter shaft 14 (and thus tapered end portions 18, 20 are generally termed cones or cone portions). The balloon 12 is sealed at balloon ends on the cone portions 18, 20 to allow the inflation of the balloon 12 via one or more inflation lumens 17 extending within catheter shaft 14 and communicating with the interior of the balloon 12.

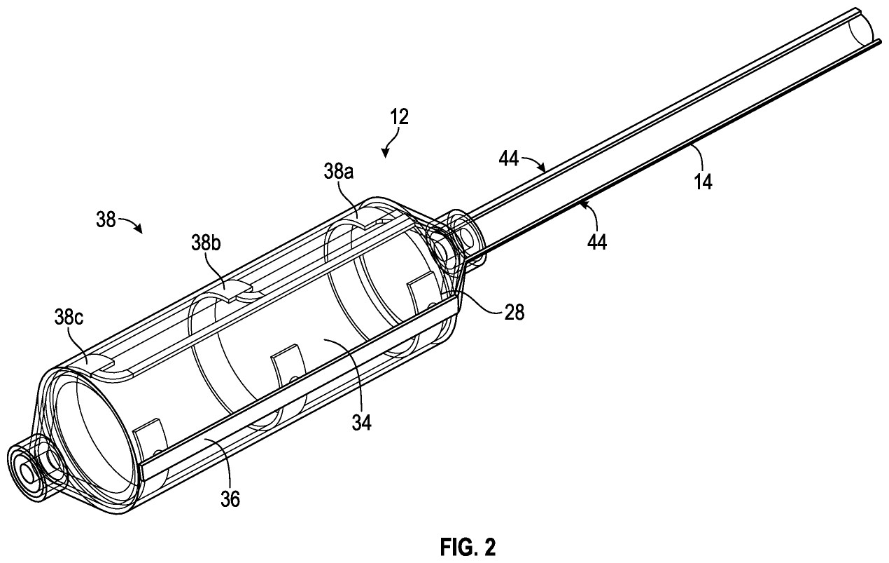

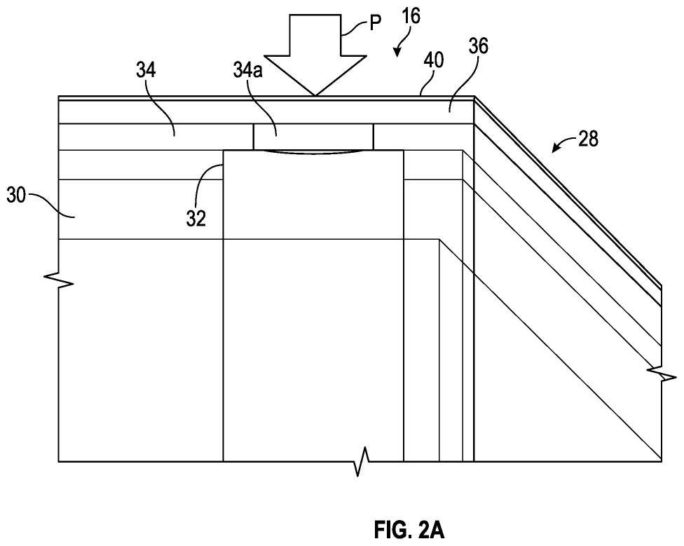

As indicated in FIG. 2, the catheter shaft 14 further includes an elongated, tubular shaft 24 at least partially nested therein. This internal shaft 24 includes a guidewire lumen 19 that directs a guidewire 26 through the catheter 10, including along the distal end portion on which the single balloon 12 is located, and out through a tip of the catheter. As illustrated in FIG. 1, this guidewire 26 may extend through the proximal end portion of the catheter 10 and through a first port 25 of a connector or hub 27 into the guidewire lumen to achieve an “over the wire” (OTW) arrangement, but could also be provided in a “rapid exchange” (RX) configuration (in which the guidewire 26 exits a lateral opening closer to the distal end, not shown) or else is fed through the tip at a passage distally of the balloon 12 (“short” RX; not shown). A second port 29 may also be associated with catheter 10, such as by way of connector or hub 27, for introducing a fluid (e.g., saline solution, a contrast agent, or both) into the interior compartment of the balloon 12 via the inflation lumen 17.

Inflatable balloon 12 may include a balloon wall 28 forming the interior for receiving the inflation fluid. The balloon 12 may be a non-compliant balloon having a balloon wall 28 that maintains its size and shape in one or more directions when the balloon is inflated, and may comprise a plurality of inelastic fibers arranged in one or more layers. The balloon 12 in such case also has a pre-determined surface area that remains constant during and, after inflation, has a pre-determined length and predetermined diameter that each, or together, remain constant during and after inflation. The balloon 12 may also be compliant (e.g., formed of an stretchable or elastic material), meaning the addition of fluid pressure causes further expansion beyond a nominal diameter (and includes semi-compliant balloons, which have a more limited degree of elasticity but thus still allow for expansion beyond a nominal diameter). The balloon 12 can have any of a variety of diameters, ranging from 1-40 mm, such as 2.0-8.0 mm, and may have any of a variety of lengths ranging from 1-300 mm. More than one inflatable balloon may be associated with the catheter 10.

According to a first aspect of the disclosure, the inflatable balloon 12 may be adapted to include an indicator for indicating contact between one or more portions of the balloon and an inner vessel wall in which the balloon is located. With reference to FIGS. 2 and 2A, the balloon 12 is formed as a multi-layered structure, including a pressure sensor for sensing the force created as a result of contact between at least one portion of the balloon on inflation and the vessel inner wall (which inner wall may comprise a lesion or other area in need of treatment using balloon dilatation).

In a first embodiment, the pressure sensor comprises one or more embedded electrical contacts on or along the intermediate portion 16 of the balloon 12. Specifically, and as perhaps best understood from FIG. 2A, the balloon wall 28 along this portion 16 includes a base balloon layer 30, which may be formed using a thin polymeric material, such as for example nylon having a thickness of 0.0005 inches. A first conductor in the form of a first contact layer 32 overlies the base balloon layer 30 and may be adhesively attached thereto. An insulator, such as in the form of an insulating layer 34 including an opening 34a, is placed over this contact layer 32, and may also be adhesively attached in place. A second conductor, which may be in the form of a second contact layer 36, includes a conductive portion applied to the insulation layer 34, such as using an adhesive, and an optional outer layer 40 may be provided. The second contact layer 36 may comprise an elongated, conductive strip of material connected via conductors, such as wires 44, extending along the shaft 14 to a source of power, as outlined further in the following description.

Thus, as can be understood from FIG. 2A, when the balloon 12 is expanded by applying fluid to an interior compartment thereof via inflation lumen 17, pressure P from the outer surface contacting the inner vessel wall presses the second contact layer 36 through the opening 34a into contact with the first contact layer 32, thus forming a circuit. By applying power to the circuit, an indication of the contact may be provided via an indicator (as outlined further below), thus forming a pressure sensor for providing the user with feedback as to the contact made between the balloon 12 and the inner wall of the vessel as a result of the pressure generated from such contact (and thus an indication that the user may halt the inflation process because of the indication that contact is being made between the balloon 12 and the inner wall of the vessel).

Referring back to FIG. 2, the first contact layer 32 may comprise one or more contact bands 38. Specifically, the balloon 12 may include a first proximal band 38a associated with a proximal end portion of the intermediate portion 16, a second intermediate band 38b associated with a middle part of the intermediate portion, and a third distal band 38c associated with a distal end portion of the intermediate portion, each for sensing the contact between the corresponding balloon portion and the inner wall of the vessel. Each band 38a, 38b, 38c may extend in a transverse or circumferential direction, and may also be connected to an elongated conductive portion extending in a longitudinal direction for connecting with associated wires 44 for forming the circuit with the second conductor, such as contact layer 36, when pressure is applied to the balloon 12.

The band(s) 38a, 38b, 38c may comprise a conductive material, such as a metal or a conductive polymer, and may be in the form of partial (e.g., C-shaped) rings or bands, which may be adhesively attached to the one or more adjacent layers forming an encapsulated arrangement. These partial or C-shaped bands 38a, 38b, 38c allow the balloon 12 to expand while providing adequate surface area for the second contact layer 36 to make suitable contact as a result of the external force supplied by the inner wall of the vessel. An alternative approach may involve applying a sputter coat the base balloon with a conductive metal (e.g., gold) to forming a very thin layer (2-20 nm) to the surface. Yet another alternative is to cut (such as by using a laser) conductive tape in a zig-zag pattern and then apply it to the balloon 12 in a similar incomplete band or ring shape.

As noted above, the insulating layer 34 includes one or more openings 34a to allow the contact layers 32, 36 to engage when sufficient pressure is applied. The pressure required may be controlled by the selection of material for the insulating layer 34, the thickness, and the size of opening 34a. The opening 34a may be circular to allow for uniform stretching when the balloon 12 is inflated. One option for the insulating layer 34 is a thin, non-conductive polymer (e.g., a PET layer having a thickness of 0.00025″).

The second contact layer 36 may comprise a single thin layer of conductive (e.g., metallic or conductive polymer) tape, which may extend the full length of the intermediate portion 16 of the balloon 12. Another option for forming this layer 36 is to sputter coat a non-conductive outer layer (e.g., Pebax or PET) with a conductive material, such as gold, to achieve a thinner layer.

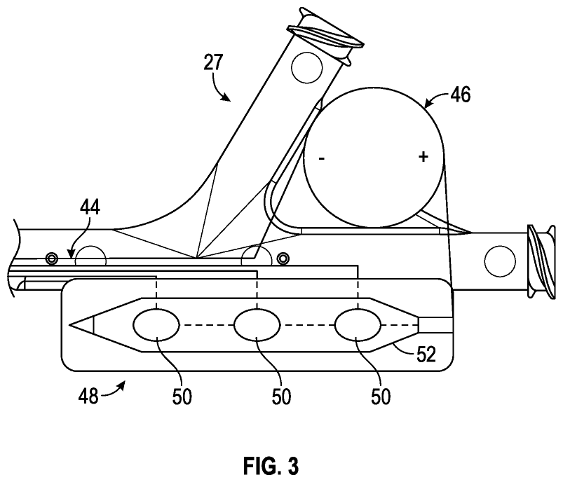

With reference to FIG. 3, wires 44 may extend proximally from the first contact layer 32 through either the inflation lumen 17 or, in a multi-lumen design, a third lumen (not shown) and to the hub 27. In one embodiment, the hub 27 includes a battery 46 and an indicator 48, such as for example or more lights 50, such as LEDs, forming part of a normally open circuit. The lights 50 thus indicate to the user when the distal, middle, and proximal bands 38a, 38b, 38c make contact with the second contact layer 36 by radial compression from the pressure created from the balloon 12 contacting the vessel wall, thus completing the associated circuit(s). An optional balloon diagram 52 may also be provided on the indicator 48 to aid the user in identifying when each section of the balloon has adequately contacted the vessel wall. As can be appreciated, the number of lights provided may vary depending on the length of the balloon (e.g., a short balloon may only need 1-2 indicator lights, whereas a 300 mm balloon may need more than three lights).

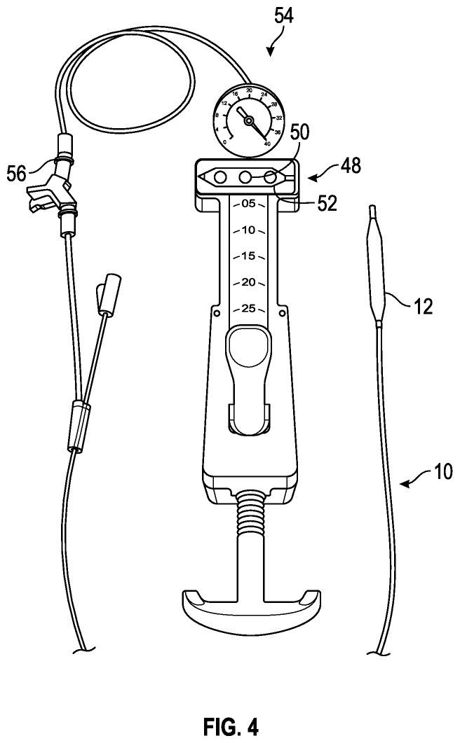

In another embodiment, and with reference to FIG. 4, the indicator 48 may be built into an indeflator 54 for inflating and deflating the balloon 12. Specifically, the indeflator 54 may house the indicator 48, which again may be in the form of lights 50, as well as a battery (not shown) to power the circuit, and may optionally include a balloon diagram 52. An electrical connection may be provided by contacts on the inflation port 25 of the hub 27 and contacts on the connector 56 (luer) of the indeflator 54. One benefit of having the indicator 48 on the indeflator 54 is that the user necessarily would be viewing the pressure gauge during inflation, and the indicator can be placed directly above or below it to provide a direct feedback as to the contact established at the point of inflation.

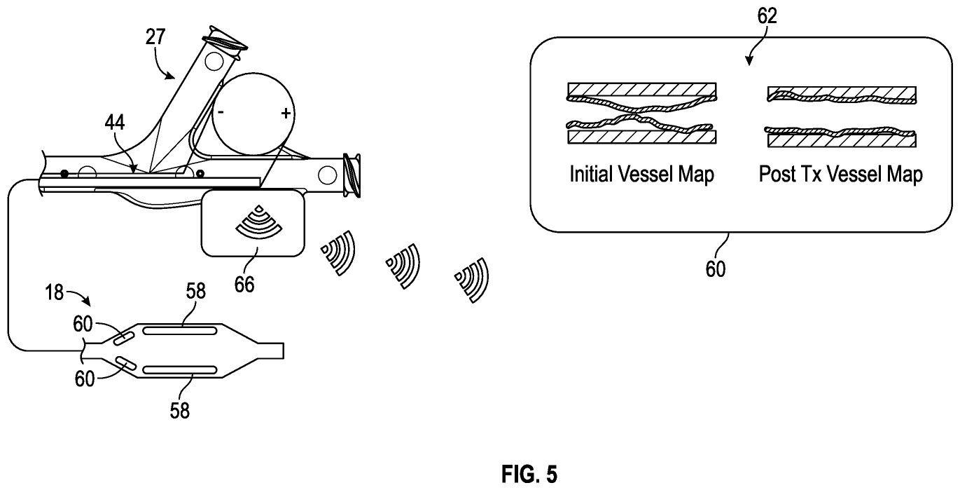

Other variations may have different types of pressure sensors for determining vessel wall contact. For example, as shown in FIG. 5, one or more closed pockets 58 of incompressible fluid may be provided between the balloon layers that interface with pressure sensors 60 in the proximal cone 18 of the balloon 12 or further proximal on the catheter. As the balloon expands and contacts the vessel wall, the pressure in the fluid pockets 58 would increase, and a transmitter 66 may via a suitable connection (wireless or wired) transmit the signal to a computer 60 including or associated with a display 62, which thus serves as the indicator in this example. These sensors 60 could then be read remotely via a wireless communication protocol (e.g., Bluetooth, Wi-Fi, etc.) or a cord to the display 62 where a visualization of the vessel would be provided based on the corresponding output signal(s).

Another variation of a pressure sensor may include one or more pieces of piezoelectric material 64 associated with the balloon 12. When the balloon 12 is thus expanded and compressed against the vessel wall, the piezoelectric material 64 generates a voltage, which may be transmitted via a transmitter 66 using a wireless or wired connection to a computer 60 including a display 62, which also serves as an indicator. The computer 60 may be programmed to convert the voltages as an input and generate a visual display of the vessel undergoing treatment. The visual display 60 may also provide a visualization of the balloon 12 and where it is experiencing different pressures based on the voltage generated from the piezoelectric material 64, with a corresponding readout as shown in FIG. 5.

The benefit of these approaches is that a variable output could be read, rather than the on/off signal from the electrical contacts. This creates an opportunity for informatics. Specifically, by collecting data on all patients who received treatments using such a balloon 12, machine learning may be employed, and treatment strategies may be optimized. Additionally, if data is collected and recorded for a specific patient, on return visits the clinician may be able to understand why a specific angioplasty treatment or drug coated balloon treatment more effective than previous treatments. Rather than relying on inflating the balloon to a specific percentage overstretch (e.g. 10%) and a specific duration (e.g. 2 min), software may be able to use the pressure readout from the indeflator 54 to achieve an optimal treatment.

An additional benefit to a visual display or readout is the ability to map the vessel during an angioplasty procedure. As the balloon 12 inflates and contacts the vessel inner wall, the pressure increases. Depending on the number of pressure sensors on the balloon 12 and the morphology of the lesion, the pressure may increase differently at different locations. These differences may be processed to generate a visual map of the vessel prior to treatment or as the balloon first makes contact with a lesion.

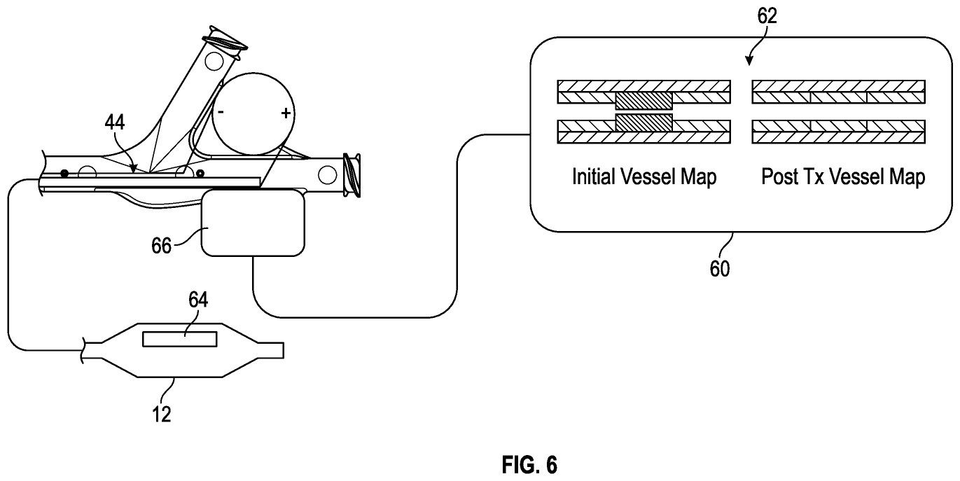

The balloon 12 may then be inflated until a desired level of overstretch occurs or until a certain pressure reading is observed on the monitor. The balloon may then remain inflated and the pressure during dwell monitored to determine whether the lesion has been appropriately dilated. Then, as the balloon is deflated, the pressure can be monitored to understand how the lesion responded to the dilatation and display a readout as to the appearance of the post-treatment vessel, as shown in FIG. 6.

Summarizing, this disclosure may be considered to relate to the following items:
1. A medical apparatus for performing a medical procedure in a vessel having an inner wall, comprising:
   an inflatable balloon including a pressure sensor for sensing contact between the inner wall of the vessel and the balloon when inflated.
2. The medical apparatus of item 1, wherein the pressure sensor comprises a first conductor for contacting a second conductor so as to form an electrical connection when the balloon is compressed against the inner wall.
3. The medical apparatus of item 2, wherein the first conductor is connected to a source of power.
4. The medical apparatus of item 2 or 3, wherein the second conductor comprises a band, which in any embodiment may be C-shaped.
5. The medical apparatus of items 2-4, wherein the first conductor comprises an elongated strip for contacting the first conductor.
6. The medical apparatus of items 2-5, wherein the first conductor is located at a proximal end portion of the inflatable balloon and further including a third conductor located at a distal end portion of the inflatable balloon for contacting the second conductor when the balloon is inflated to contact an inner wall of the vessel.
7. The medical apparatus of item 6, further including a fourth conductor located along an intermediate portion of the balloon for contacting the second conductor when the balloon is inflated to contact an inner wall of the vessel.
8. The medical apparatus of any of the foregoing items, wherein the first and second conductors are provided in a wall of the balloon.
9. The medical apparatus of item 8, wherein the wall includes an insulator for separating the first and second conductors when the balloon is not in contact with the inner wall of the vessel, the insulator including an opening for allowing the first and second conductors to make contact as a result of the balloon when inflated pressing against the inner wall of the vessel.
10. The medical apparatus of any of items 2-9, further including an indicator for indicating when the first and second conductors are in contact.
11. The medical apparatus of item 10, further including a shaft for supporting the balloon, the shaft including a proximal hub including the indicator.
12. The medical apparatus of item 10, further including an inflator for inflating the balloon, the inflator including the indicator.
13. The medical apparatus of any of items 10-12, wherein the balloon includes a plurality of first conductors, the indicator indicating when each of the first conductors forms an electrical connection with the second conductor.
14. The medical apparatus of any of items 10-13, further including a shaft for supporting the balloon, the shaft including a proximal hub adapted for transmitting a signal indicating the electrical connection between the first conductor and the second conductor.
15. The medical apparatus of item 14, wherein the proximal hub is adapted for wirelessly transmitting the signal.
16. The medical apparatus of item 1 or any of items 10-15, wherein the pressure sensor comprises a pocket of fluid within a wall of the balloon.
17. The medical apparatus of item 1 or any of items 10-15, wherein the pressure sensor comprises a piezoelectric material connected to a wall of the balloon.
18. A medical apparatus for performing a medical procedure in a vessel having an inner wall, comprising:
   an inflatable balloon comprising a first sensor along a first portion of the balloon for sensing contact between the first portion of the balloon when inflated and the inner wall of the vessel, and a second sensor along a second portion of the balloon for sensing contact between the second portion of the balloon when inflated and the inner wall of the vessel.
19. The medical apparatus of item 18, wherein the first sensor is located along a proximal end portion of the balloon and the second sensor is located along a distal end portion of the balloon.
20. The medical apparatus of items 18-19, further including a third sensor between the first and second sensors for sensing contact between a third portion of the balloon when inflated and the inner wall of the vessel.
21. The medical apparatus of items 18-20, wherein the first sensor comprises a first conductor for contacting a second conductor so as to form an electrical connection when the first portion balloon is inflated to contact the inner wall of the vessel.
22. The medical apparatus of item 21, wherein the second sensor comprises a third conductor for contacting the second conductor so as to form an electrical connection when the second portion balloon is inflated to contact the inner wall of the vessel.
23. A medical apparatus for performing a medical procedure in a vessel having an inner wall, comprising:
   an inflatable balloon comprising a first sensor along a first portion of the balloon for sensing contact between the first portion of the balloon when inflated and the inner wall of the vessel, and
   an indicator, such as a light or display, for indicating when the first portion of the inflatable balloon is in contact with the inner wall of the vessel.
24. The medical apparatus of item 23, further including a catheter shaft having a proximal hub including the indicator.
25. The medical apparatus of item 23 or item 24, further including a catheter shaft having a proximal hub including adapted for transmitting a signal from the first sensor.
26. The medical apparatus of item 25, wherein the proximal hub is adapted for wirelessly transmitting the signal.
27. The medical apparatus of item 23, wherein the first sensor is located along a proximal end portion of the balloon and further including a second sensor is located along a distal end portion of the balloon.
28. The medical apparatus of item 27, further including a third sensor between the first and second sensors for sensing contact between a third portion of the balloon when inflated and the inner wall of the vessel.
29. The medical apparatus of item 23, wherein the first sensor comprises a first conductor for contacting a second conductor so as to form an electrical connection when the first portion balloon is inflated to contact the inner wall of the vessel.
30. The medical apparatus of item 29, further including a second sensor comprising a third conductor for contacting the second conductor so as to form an electrical connection when the second portion balloon is inflated to contact the inner wall of the vessel.
31. A method for evaluating an inner wall of a vessel, comprising:
   expanding a medical balloon including at least one sensor for sensing contact between a portion of the balloon and the inner wall; and
   mapping the inner wall based on the contact sensed by the at least one sensor.
32. The method of item 31, wherein the mapping comprises visualizing the inner wall of the vessel.
33. The method of item 31, wherein the visualizing comprises visualizing the inner wall of the vessel in a first inflation state of the medical balloon and visualizing the inner wall of the vessel in a second inflation state of the balloon.
34. A method of performing a medical procedure in a vessel having an inner wall, comprising:
   inflating a balloon in the vessel; and
   indicating contact between a plurality of portions of the balloon and the inner wall of the vessel.
35. The method of item 34, wherein the indicating step comprises providing a first visual indication of contact between a first portion of the balloon and the inner wall, and a second visual indication of contact between a second portion of the balloon and the inner wall.
36. The method of item 35, wherein the step of providing the first and second visual indications comprises illuminating first and second lights on an indicator attached to a shaft supporting the balloon.

As used herein, the following terms have the following meanings:
“A”, “an”, and “the” as used herein refers to both singular and plural referents unless the context clearly dictates otherwise. By way of example, “a compartment” refers to one or more than one compartment.
“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.
“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., component or the like does not exclude or preclude the presence of additional, non-recited components, features, element, members, steps, known in the art or disclosed therein.

Although the invention is described in conjunction with specific embodiments, many alternatives, modifications, and variations will be apparent to those skilled in the art. Accordingly, this disclosure embraces all such alternatives, modifications, and variations that fall within the spirit and scope of the appended claims. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. A medical apparatus for performing a medical procedure in a vessel having an inner wall, comprising:
an inflatable balloon including a pressure sensor for sensing contact between the inner wall of the vessel and the balloon when inflated.

2. The medical apparatus of claim 1, wherein the pressure sensor comprises a first conductor and a second conductor, the first conductor arranged for contacting the second conductor so as to form an electrical connection when the balloon is compressed against the inner wall.

3. The medical apparatus of claim 2, wherein the first conductor is connected to a source of power.

4. The medical apparatus of claim 3, wherein the second conductor comprises a band.

5. The medical apparatus of claim 3, wherein the first conductor comprises an elongated strip for contacting the second conductor.

6. The medical apparatus of claim 5, wherein the first conductor is located at a proximal end portion of the inflatable balloon and further including a third conductor located at a distal end portion of the inflatable balloon for contacting the second conductor when the balloon is inflated to contact an inner wall of the vessel.

7. The medical apparatus of claim 6, further including a fourth conductor located along an intermediate portion of the balloon for contacting the second conductor when the balloon is inflated to contact an inner wall of the vessel.)

8. The medical apparatus of claim 1, wherein the first and second conductors are provided in a wall of the balloon.

9. The medical apparatus of claim 8, wherein the wall includes an insulator for separating the first and second conductors when the balloon is not in contact with the inner wall of the vessel, the insulator including an opening for allowing the first and second conductors to make contact as a result of the balloon when inflated pressing against the inner wall of the vessel.

10. The medical apparatus of claim 2, further including an indicator for indicating when the first and second conductors are in contact.

11. The medical apparatus of claim 10, further including a shaft for supporting the balloon, the shaft including a proximal hub including the indicator.

12. The medical apparatus of claim 10, further including an inflator for inflating the balloon, the inflator including the indicator.

13. The medical apparatus of claim 12, wherein the balloon includes a plurality of first conductors, the indicator indicating when each of the first conductors forms an electrical connection with the second conductor.

14. The medical apparatus of claim 10, further including a shaft for supporting the balloon, the shaft including a proximal hub adapted for transmitting a signal indicating the electrical connection between the first conductor and the second conductor.

15. The medical apparatus of claim 14, wherein the proximal hub is adapted for wirelessly transmitting the signal.

16. The medical apparatus of claim 1, wherein the pressure sensor comprises a pocket of fluid within a wall of the balloon.

17. The medical apparatus of claim 1, wherein the pressure sensor comprises a piezoelectric material connected to a wall of the balloon.

18. A medical apparatus for performing a medical procedure in a vessel having an inner wall, comprising:
an inflatable balloon comprising a first sensor on a first portion of the balloon for sensing contact between the first portion of the balloon when inflated and the inner wall of the vessel, and a second sensor on a second portion of the balloon for sensing contact between the second portion of the balloon when inflated and the inner wall of the vessel.

19. The medical apparatus of claim 18, wherein the first sensor is located along a proximal end portion of the balloon and the second sensor is located along a distal end portion of the balloon.

20. The medical apparatus of claim 19, further including a third sensor between the first and second sensors for sensing contact between a third portion of the balloon when inflated and the inner wall of the vessel.

21-36. (canceled)
